(19) United States
(12) Patent Application Publication
(10) Pub. No.: US 2024/0228438 A1
(43) Pub. Date: Jul. 11, 2024

(54) SOLID FORMS OF SALTS OF 4-[5-[(3S)-3-AMINOPYRROLIDINE-1-CARBONYL]-2-[2-FLUORO-4-(2- HYDROXY-2-ETHYLPROPYL)PHENYL]PHENYL]-2-FLUORO-BENZONITRILE

(71) Applicants: Astex Pharmaceuticals, Inc., Pleasanton, CA (US); Taiho Pharmaceutical Co., Ltd., Tokyo (JP)

(72) Inventors: Nipun Davar, Pleasanton, CA (US); Matthew Johnson, Raleigh, NC (US); Suresh Manthati, Sunnyvale, CA (US); Gemma Zeund, Termen, Valais, CA (US)

(21) Appl. No.: 18/560,360

(22) PCT Filed: May 10, 2022
(86) PCT No.: PCT/US2022/028606

§ 371 Date: Nov. 10, 2023

Related U.S. Application Data
(60) Provisional application No. 63/187,125, filed on May 11, 2021.

(51) Int. Cl. C07D 207/14; A61K 31/40

(52) U.S. Cl. CPC C07D 207/14; A61K 31/40

(57) ABSTRACT

Forms of 4-[5-[(3S)-3-aminopyrrolidine-1-carbonyl]-2-[2-fluoro-4-(2-hydroxy-2-methyl-propyl)phenyl]phenyl]-2-fluoro-benzonitrile, designated herein as Compound I, were prepared and characterized in the solid state. Also provided are processes of manufacture and methods of using the forms of Compound I.

### CROSS REFERENCE TO RELATED APPLICATIONS

This application claims the benefit under 35 U.S.C. § 119(e) of U.S. Provisional Application No. 63/187,125 filed May 11, 2021, which is incorporated herein by reference in its entirety.

### FIELD

The present disclosure relates generally to solid forms of compounds that modulate lysine-specific histone demethylase 1A (LSD1), pharmaceutical compositions thereof, therapeutic uses thereof, and processes for making the solid forms.

### BACKGROUND

The compound 4-[5-[(3S)-3-aminopyrrolidine-1-carbonyl]-2-[2-fluoro-4-(2-hydroxy-2-methyl-propyl)phenyl]phenyl]-2-fluoro-benzonitrile (referred to herein as Compound I) is a potent LSD1 inhibitor and used as an antitumor agent or an agent for preventing and/or treating cancer.

It is desired that such an LSD1 inhibitor shows stability when it is used for a pharmaceutical formulation.

Also, there is a desire to develop such an LSD1 inhibitor that can be easily handled. It is known that the hygroscopicity of a biologically active compound affects the handling of the compound during its incorporation into a potential pharmaceutical composition. Hygroscopic compounds present problems due to their moisture absorption which leads to variations in compound mass depending on the amount of water present in the surrounding environment, making it difficult to accurately evaluate the compound's biological efficacy and to ensure the uniformity of pharmaceutical compositions containing the compound. Further hygroscopic compounds can be sticky causing problems during processing. Therefore, a solid form with low hygroscopicity and/or stickiness is desirable.

### SUMMARY

The present disclosure provides polymorphic and/or amorphous forms of Compound I and salts, co-crystals, solvates, and hydrates thereof. Also described herein are processes for making the forms of Compound I, pharmaceutical compositions comprising forms of Compound I, and methods for using such forms and pharmaceutical compositions in the treatment of diseases mediated by LSD1.

### BRIEF DESCRIPTION OF THE DRAWINGS

FIG. 1 is an X-ray powder diffractogram (XRPD) of Compound I Form I.

FIG. 2 is an X-ray powder diffractogram of Compound I Form II.

FIG. 3A is an X-ray powder diffractogram of Compound I Form IIIA. FIG. 3B is a differential scanning calorimeter (DSC) curve of Compound I Form III.

FIG. 4A is an X-ray powder diffractogram of Compound I Form IVA. FIG. 4B is a differential scanning calorimeter (DSC) curve of Compound I Form IV.

FIG. 5A is an X-ray powder diffractogram of Compound I Form V. FIG. 5B is a differential scanning calorimeter (DSC) curve of Compound I Form V.

FIG. 6A is an X-ray powder diffractogram of Compound I Form VI. FIG. 6B is a polarized light microscopy (PLM) image of Compound I Form VII.

FIG. 7A is a PLM image of Compound I Form I. FIG. 7B is a PLM image of Compound I Form II.

FIG. 8A is an X-ray powder diffractogram of Compound I Form IIIB. FIG. 8B is an X-ray powder diffractogram of Compound I Form IIIC.

FIG. 9A is an X-ray powder diffractogram of Compound I Form IIID.

FIG. 10A is a differential scanning calorimeter (DSC) curve of Compound I Form IVB. FIG. 10B is an X-ray powder diffractogram of Compound I Form IVB.

### DETAILED DESCRIPTION

The compound 4-[5-[(3S)-3-aminopyrrolidine-1-carbonyl]-2-[2-fluoro-4-(2-hydroxy-2-methyl-propyl)phenyl]phenyl]-2-fluoro-benzonitrile, designated herein as Compound I, has the following formula:

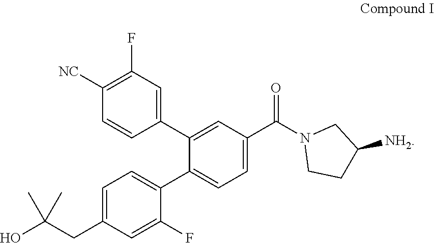 [SMILES: CC(C)(O)Cc1ccc(-c2ccc(C(=O)N3CC[C@H](N)C3)cc2-c2ccc(C#N)c(F)c2)c(F)c1]

Compound I is an inhibitor of LSD1. The synthesis and method of use thereof is described in PCT International Application Publication No. WO 2017/090756 which is herein incorporated by reference in its entirety.

### 1. Definitions

As used in the present specification, the following words and phrases are generally intended to have the meanings as set forth below, except to the extent that the context in which they are used indicates otherwise.

The term “comprise” and variations thereof, such as, “comprises” and “comprising” are to be construed in an open, inclusive sense, that is, as “including, but not limited to.” Further, the singular forms “a,” “an,” and “the” include plural references unless the context clearly dictates otherwise. Thus, reference to “the compound” includes a plurality of such compounds, and reference to “the assay” includes reference to one or more assays and equivalents thereof known to those skilled in the art.

Reference to “about” a value or parameter herein includes (and describes) embodiments that are directed to that value or parameter per se. In certain embodiments, the term “about” includes the indicated amount ±10%. In other embodiments, the term “about” includes the indicated amount ±5%. In certain other embodiments, the term “about” includes the indicated amount ±2.5%. In certain other embodiments, the term “about” includes the indicated amount ±1%. Also, to the term “about X” includes description of “X”.

Recitation of numeric ranges of values throughout the disclosure is intended to serve as a shorthand notation of referring individually to each separate value falling within the range inclusive of the values defining the range, and each separate value is incorporated in the specification as it were individually recited herein.

Forms of Compound I or salts, co-crystals, solvates, or hydrates thereof are provided herein. In one embodiment, reference to a form of Compound I or a salt, co-crystal, solvate, or hydrate thereof means that at least 50% to 99% (e.g., at least 50%, at least 55%, at least 60%, at least 65%, at least 70%, at least 75%, at least 80%, at least 85%, at least 90%, at least 95%, or at least 99%) of Compound I or a salt, co-crystal, solvate, or hydrate thereof present in a composition is in the designated form. For instance, in one embodiment, reference to Compound I Form I means that at least 50%, at least 55%, at least 60%, at least 65%, at least 70%, at least 75%, at least 80%, at least 85%, at least 90%, at least 95%, or at least 99% of Compound I present in a composition is in Form I.

The term “solid form” refers to a type of solid-state material that includes amorphous as well as crystalline forms. The term “crystalline form” refers to polymorphs as well as solvates, hydrates, etc. The term “polymorph” refers to a particular crystal structure having particular physical properties such as X-ray diffraction, melting point, and the like.

The term “co-crystal” refers to a molecular complex of a compound disclosed herein and one or more non-ionized co-crystal formers connected via non-covalent interactions. In some embodiments, the co-crystals disclosed herein may include a non-ionized form of Compound I (e.g., Compound I free form) and one or more non-ionized co-crystal formers, where non-ionized Compound I and the co-crystal former(s) are connected through non-covalent interactions. In some embodiments, co-crystals disclosed herein may include an ionized form of Compound I (e.g., a salt of Compound I) and one or more non-ionized co-crystals formers, where ionized Compound I and the co-crystal former(s) are connected through non-covalent interactions. Co-crystals may additionally be present in anhydrous, solvated or hydrated forms. In certain instances, co-crystals may have improved properties as compared to the parent form (i.e., the free molecule, zwitterion, etc.) or a salt of the parent compound. Improved properties can be increased solubility, increased dissolution, increased bioavailability, increased dose response, decreased hygroscopicity, increased stability, a crystalline form of a normally amorphous compound, a crystalline form of a difficult to salt or unsaltable compound, decreased form diversity, more desired morphology, and the like. Methods for making and characterizing co-crystals are known to those of skill in the art.

The term “co-crystal former” or “co-former” refers to one or more pharmaceutically acceptable bases or pharmaceutically acceptable acids disclosed herein in association with Compound I, or any other compound disclosed herein.

The term “solvate” refers to a complex formed by combination of solvent molecules with molecules or ions of the solute. The solvent can be an organic compound, an inorganic compound, or a mixture of both. As used herein, the term “solvate” includes a “hydrate” (i.e., a complex formed by combination of water molecules with molecules or ions of the solute), hemi-hydrate, channel hydrate, etc. Some examples of solvents include, but are not limited to, methanol, N,N-dimethylformamide, tetrahydrofuran, dimethylsulfoxide, and water. In general, the solvated forms are equivalent to unsolvated forms and are encompassed within the scope of the present disclosure.

The term “desolvated” refers to a Compound I form that is a solvate as described herein, and from which solvent molecules have been partially or completely removed. Desolvation techniques to produce desolvated forms include, without limitation, exposure of a Compound I form (solvate) to a vacuum, subjecting the solvate to an elevated temperature, exposing the solvate to a stream of gas, such as air or nitrogen, or any combination thereof. Thus, a desolvated Compound I form can be anhydrous, i.e., completely without solvent molecules, or partially solvated wherein solvent molecules are present in stoichiometric or non-stoichiometric amounts.

The term “amorphous” refers to a state in which the material lacks long range order at the molecular level and, depending upon temperature, may exhibit the physical properties of a solid or a liquid. Typically such materials do not give distinctive X-ray diffraction patterns and, while exhibiting the properties of a solid, are more formally described as a liquid. Upon heating, a change from solid to liquid properties occurs which is characterized by a change of state, typically second order (glass transition).

Any formula or structure given herein, including Compound I, is also intended to represent unlabeled forms as well as isotopically labeled forms of the compounds. It is understood that for any given atom, the isotopes may be present essentially in ratios according to their natural occurrence, or one or more particular atoms may be enhanced with respect to one or more isotopes using synthetic methods known to one skilled in the art. Thus, hydrogen includes for example ¹H, ²H, ³H; carbon includes for example ¹¹C, ¹²C, ¹³C, ¹⁴C; oxygen includes for example ¹⁶O, ¹⁷O, ¹⁸O; nitrogen includes for example ¹³N, ¹⁴N, ¹⁵N; sulfur includes for example ³²S, ³³S, ³⁴S, ³⁵S, ³⁶S, ³⁷S, ³⁸S; fluoro includes for example ¹⁷F, ¹⁸F, ¹⁹F; chloro includes for example ³⁵Cl, ³⁶Cl, ³⁷Cl, ³⁸Cl, ³⁹Cl; and the like.

As used herein, the terms “treat,” “treating,” “therapy,” “therapies,” and like terms refer to the administration of material, e.g., any one or more solid, crystalline or polymorphs of Compound I as described herein in an amount effective to prevent, alleviate, or ameliorate one or more symptoms of a disease or condition, i.e., indication, and/or to prolong the survival of the subject being treated.

The term “administering” refers to oral administration, administration as a suppository, topical contact, intravenous, intraperitoneal, intramuscular, intralesional, intranasal or subcutaneous administration, or the implantation of a slow-release device e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc.

As used herein, the term “modulating” or “modulate” refers to an effect of altering a biological activity, especially a biological activity associated with a particular biomolecule such as LSD1. For example, an agonist or antagonist of a particular biomolecule modulates the activity of that biomolecule, e.g., LSD1, by either increasing (e.g. agonist, activator), or decreasing (e.g. antagonist, inhibitor) the activity of the biomolecule. Such activity is typically indicated in terms of an inhibitory concentration (IC₅₀) or excitation concentration (EC₅₀) of the compound for an inhibitor or activator, respectively, with respect to, for example, LSD1.

As used herein, the term “LSD1 mediated disease or condition,” refers to a disease or condition in which the biological function of LSD1, including any mutations thereof, affects the development, course, and/or symptoms of the disease or condition, and/or in which modulation of LSD1 alters the development, course, and/or symptoms of the disease or condition. The LSD1 mediated disease or condition includes a disease or condition for which LSD1 modulation provides a therapeutic benefit, e.g. wherein treatment with compound(s), including one or more solid, crystalline or polymorphs of Compound I as described herein, provides a therapeutic benefit to the subject suffering from or at risk of the disease or condition.

As used herein, the term “composition” refers to a pharmaceutical preparation suitable for administration to an intended subject for therapeutic purposes that contains at least one pharmaceutically active compound, including any solid form thereof. The composition may include at least one pharmaceutically acceptable component to provide an improved formulation of the compound, such as a suitable carrier or excipient.

As used herein, the term “subject” or “patient” refers to a living organism that is treated with compounds as described herein, including, but not limited to, any mammal, such as a human, other primates, sports animals, animals of commercial interest such as cattle, farm animals such as horses, or pets such as dogs and cats.

The term “pharmaceutically acceptable” indicates that the indicated material does not have properties that would cause a reasonably prudent medical practitioner to avoid administration of the material to a patient, taking into consideration the disease or conditions to be treated and the respective route of administration. For example, it is commonly required that such a material be essentially sterile, e.g., for injectables.

In the present context, the term “therapeutically effective” or “effective amount” indicates that the materials or amount of material is effective to prevent, alleviate, or ameliorate one or more symptoms of a disease or medical condition, and/or to prolong the survival of the subject being treated. The therapeutically effective amount will vary depending on the compound, the disorder or condition and its severity and the age, weight, etc., of the mammal to be treated. For example, an effective amount is an amount sufficient to effectuate a beneficial or desired clinical result. The effective amounts can be provided all at once in a single administration or in fractional amounts that provide the effective amount in several administrations. The precise determination of what would be considered an effective amount may be based on factors individual to each subject, including their size, age, injury, and/or disease or injury being treated, and amount of time since the injury occurred or the disease began. One skilled in the art will be able to determine the effective amount for a given subject based on these considerations which are routine in the art.

In some embodiments, the phrase “substantially shown in Figure” as applied to an X-ray powder diffractogram is meant to include a variation of ±0.2° 2θ or ±0.1° 2θ, as applied to DSC thermograms is meant to include a variation of ±3° Celsius, and as applied to thermogravimetric analysis (TGA) is meant to include a variation of ±2% in weight loss.

“Substantially pure form (of a polymorph),” in some embodiments, means that in the referenced material, at least 99.9% of the material is the referenced polymorph. “Substantially pure form (of a polymorph),” in some embodiments, means that in the referenced material, at least 99.5% of the material is the referenced polymorph. “Substantially pure form (of a polymorph),” in some embodiments, means that in the referenced material, at least 99% of the material is the referenced polymorph. “Substantially pure form (of a polymorph),” in some embodiments, means that in the referenced material, at least 98% of the material is the referenced polymorph. “Substantially pure form (of a polymorph),” in some embodiments, means that in the referenced material, at least 97% of the material is the referenced polymorph. “Substantially pure form (of a polymorph),” in some embodiments, means that in the referenced material, at least 96% of the material is the referenced polymorph. “Substantially pure form (of a polymorph),” in some embodiments, means that in the referenced material, at least 95% of the material is the referenced polymorph. In the context of the use, testing, or screening of compounds that are or may be modulators, the term “contacting” means that the compound(s) are caused to be in sufficient proximity to a particular molecule, complex, cell, tissue, organism, or other specified material that potential binding interactions and/or chemical reaction between the compound and other specified material can occur.

In addition, abbreviations as used herein have respective meanings as follows:

| | |
|---|---|
| ACN or MeCN | acetonitrile |
| DCM | dichloromethane |
| DMAc | dimethylacetamide |
| DMSO | dimethylsulfoxide |
| DSC | differential scanning calorimetry |
| DVS | dynamic vapor sorption |
| EtOAc | ethyl acetate |
| EtOH | ethanol |
| IPA | isopropyl alcohol or 2-propanol |
| IPAc | isopropyl acetate |
| LC/MS | Liquid chromatography mass spectrometry |
| MeOH | methanol |
| MEK | methyl ethyl ketone |
| MTBE | methyl tert-butyl ether |
| NMR | Nuclear magnetic resonance spectroscopy |
| nPrOH | n-propanol |
| RT | room temperature |
| TGA | thermogravimetric analysis |
| THF | tetrahydrofuran |
| v/v | volume to volume |
| w/w | weight to weight |
| XRPD | X-ray powder diffraction |

### 2. Forms of Compound I

As described generally above, the present disclosure provides crystalline forms of the compound, 4-[5-[(3S)-3-aminopyrrolidine-1-carbonyl]-2-[2-fluoro-4-(2-hydroxy-2-methyl-propyl)phenyl]phenyl]-2-fluoro-benzonitrile (hereinafter “compound” of “Compound I”), and salts, co-crystals, solvates, or hydrates thereof. Crystalline forms of Compound I and salts, co-crystals, solvates, or hydrates thereof, and other forms (e.g., amorphous forms) of Compound I and salts, co-crystals, solvates, or hydrates thereof are collectively referred to herein as “forms of Compound I.”

In some embodiments, Compound I is a free base. In some embodiments, Compound I is a salt or a co-crystal. In some embodiments, Compound I is a pharmaceutically acceptable salt or co-crystal. In some embodiments, Compound I is a solvate. In some embodiments, Compound I is a hydrate. In some embodiments, Compound I is an anhydrate.

In some embodiments, Compound I is in an amorphous form.

Techniques for characterizing crystalline forms and amorphous forms include, but are not limited to, thermal gravimetric analysis (TGA), differential scanning calorimetry (DSC), X-ray powder diffractometry (XRPD), single-crystal X-ray diffractometry, vibrational spectroscopy, e.g., infrared (IR) and Raman spectroscopy, solid-state and solution nuclear magnetic resonance (NMR) spectroscopy, optical microscopy, hot stage optical microscopy, scanning electron microscopy (SEM), electron crystallography and quantitative analysis, particle size analysis (PSA), surface area analysis, solubility measurements, dissolution measurements, elemental analysis, and Karl Fischer analysis. Characteristic unit cell parameters may be determined using one or more techniques such as, but not limited to, X-ray diffraction and neutron diffraction, including single-crystal diffraction and powder diffraction. Techniques useful for analyzing powder diffraction data include profile refinement, such as Rietveld refinement, which may be used, e.g., to analyze diffraction peaks associated with a single phase in a sample comprising more than one solid phase. Other methods useful for analyzing powder diffraction data include unit cell indexing, which allows one skilled in the art to determine unit cell parameters from a sample comprising crystalline powder.

### Compound I Form I—Hemi-Fumarate

In one embodiment, provided herein is crystalline 4-[5-[(3S)-3-aminopyrrolidine-1-carbonyl]-2-[2-fluoro-4-(2-hydroxy-2-methylpropyl)phenyl]phenyl]-2-fluoro-benzonitrile hemi fumarate (Compound I Form I), characterized by an X-ray powder diffraction pattern comprising the following peaks: 9.37°±0.2°, 14.63°±0.2° and 21.27±0.2°, expressed as 2θ angles determined using Cu K-α (λ=1.5406 Å) radiation.

In some embodiments, the diffraction pattern for Compound I Form I further comprises one or more peaks selected from 4.73°±0.2°, 18.86°±0.2°, and 20.65°±0.2°, expressed as 2θ angles determined using Cu K-α (λ=1.5406 Å) radiation.

In some embodiments, the diffraction pattern for Compound I Form I further comprises two or more peaks selected from 4.73°±0.2°, 8.18°±0.2°, 14.79°±0.2°, 18.86°±0.2°, 20.41°±0.2° and 20.65°±0.2°, expressed as 2θ angles determined using Cu K-α (λ=1.5406 Å) radiation.

In some embodiments, the diffraction pattern for Compound I Form I comprises peaks selected from 4.73°±0.2°, 8.18°±0.2°, 9.37°±0.2°, 14.63°±0.2°, 14.79°±0.2°, 18.86°±0.2°, 20.41°±0.2°, 21.27°±0.2° and 20.65°±0.2°, expressed as 2θ angles determined using Cu K-α (λ=1.5406 Å) radiation

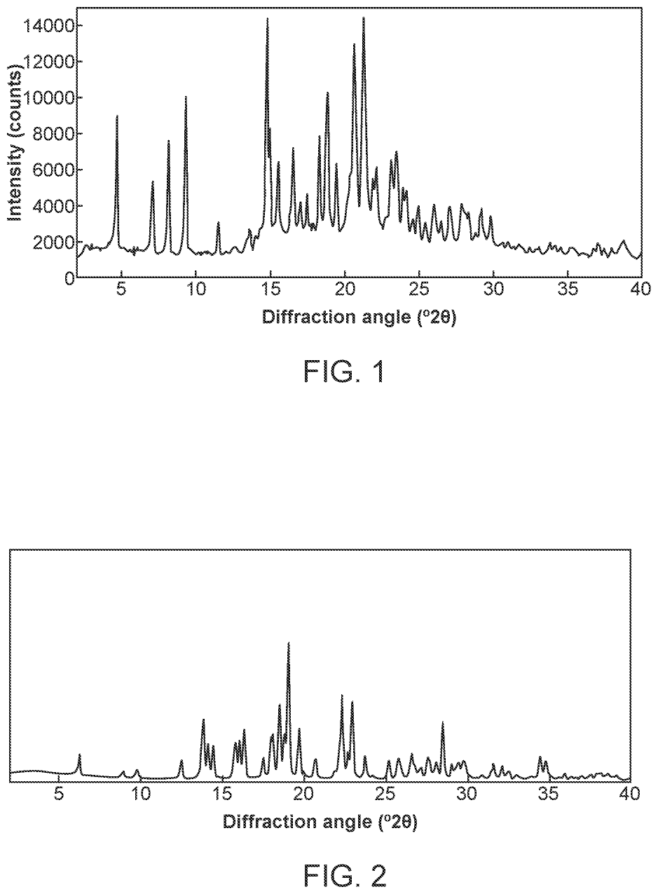

In some embodiments, the diffraction pattern for Compound I Form I is substantially as shown in FIG. 1.

In some embodiments, Compound I Form I is prepared by a process comprising
contacting a solution of Compound I in a solvent with a solution of fumaric acid in a solvent at room temperature to obtain a slurry;
warming the slurry to a temperature of about 50° C.;
cooling the slurry to room temperature;
filtering the solids from the slurry to obtain a filter-cake; and
drying the filter-cake to obtain Compound I Form I.

In some embodiments, the solvent is selected from acetonitrile, anisole, butanol, isopropanol, methyl tert-butyl ether (MTBE), ethanol, ethyl acetate, heptane, isopropyl acetate, methyl acetate, methyl ethyl ketone (MEK), and toluene. In some embodiments, the solvent is isopropanol. In some embodiments, Compound I Form I is prepared by the sequence of steps described in the Examples section.

### Compound I Form II—Mono-Oxalate

In one embodiment, provided herein is crystalline 4-[5-[(3S)-3-aminopyrrolidine-1-carbonyl]-2-[2-fluoro-4-(2-hydroxy-2-methylpropyl)phenyl]phenyl]-2-fluoro-benzonitrile mono-oxalate (Compound I Form II), characterized by an X-ray powder diffraction pattern comprising the following peaks: 13.86°±0.2°, 19.05°±0.2° and 22.94°±0.2°, expressed as 2θ angles determined using Cu K-α (λ=1.5406 Å) radiation.

In some embodiments, the diffraction pattern for Compound I Form II further comprises one or more peaks selected from 18.50°±0.2°, 22.31°±0.2°, and 28.48°±0.2°, expressed as 2θ angles determined using Cu K-α (λ=1.5406 Å) radiation.

In some embodiments, the diffraction pattern for Compound I Form II further comprises two or more peaks selected from 15.83°±0.2°, 16.32°±0.2°, 18.500±0.2°, 18.79°±0.2°, 22.31°±0.2°, and 28.48°±0.2°, expressed as 2θ angles determined using Cu K-α (λ=1.5406 Å) radiation.

In some embodiments, the diffraction pattern for Compound I Form II comprises peaks selected from 13.86°±0.2°, 15.83°±0.2°, 16.32°±0.2°, 18.50°±0.2°, 18.79°±0.2°, 19.05°±0.2°, 22.31° 40.2°, 22.94°±0.2°, and 28.48°±0.2°, expressed as 2θ angles determined using Cu K-α (λ=1.5406 Å) radiation.

In some embodiments, the diffraction pattern for Compound I Form II is substantially as shown in FIG. 2.

In some embodiments, the Compound I Form II is prepared by a process comprising
contacting a solution of Compound I in a solvent with a solution of oxalic acid in a solvent at room temperature to obtain a slurry;
warming the slurry to a temperature of about 50° C.;
cooling the slurry to room temperature;
filtering the solids from the slurry to obtain a filter-cake;
drying the filter-cake to obtain Compound I mono-oxalate salt; and
recrystallizing the Compound I mono-oxalate salt from ethanol to obtain Compound I Form II.

In some embodiments, the solvent is selected from acetonitrile, anisole, butanol, isopropanol, methyl tert-butyl ether (MTBE), ethanol, ethyl acetate, heptane, isopropyl acetate, methyl acetate, methyl ethyl ketone (MEK), and toluene. In some embodiments, the solvent is isopropanol. In some embodiments, Compound I Form II is prepared by the sequence of steps described in the Examples section.

### Compound I Form IIIA-IIID—Mesylate

In one embodiment, provided herein is crystalline 4-[5-[(3S)-3-aminopyrrolidine-1-carbonyl]-2-[2-fluoro-4-(2-hydroxy-2-methylpropyl)phenyl]phenyl]-2-fluoro-benzonitrile mesylate (Compound I Form IIIA), characterized by an X-ray powder diffraction pattern comprising the following peaks: 6.24°±0.2°, 16.47°±0.2° and 21.20°±0.2°, expressed as 2θ angles determined using Cu K-α (λ=1.5406 Å) radiation.

In some embodiments, the diffraction pattern for Compound I Form IIIA further comprises one or more peaks selected from 15.39°±0.2°, 16.97°±0.2°, and 21.51°±0.2°, expressed as 2θ angles determined using Cu K-α (λ=1.5406 Å) radiation.

In some embodiments, the diffraction pattern for Compound I Form IIIA further comprises two or more peaks selected from 8.52°±0.2°, 15.39°±0.2°, 16.97°±0.2°, 17.95°±0.2°, and 21.51°±0.2°, expressed as 2θ angles determined using Cu K-α (λ=1.5406 Å) radiation.

In some embodiments, the diffraction pattern for Compound I Form IIIA comprises peaks selected from 6.24°±0.2°, 8.52°±0.2°, 15.39°±0.2°, 16.47°±0.2°, 16.970±0.2°, 17.95°±0.2°, 21.20°±0.2°, and 21.51±0.2°, expressed as 2θ angles determined using Cu K-α (λ=1.5406 Å) radiation.

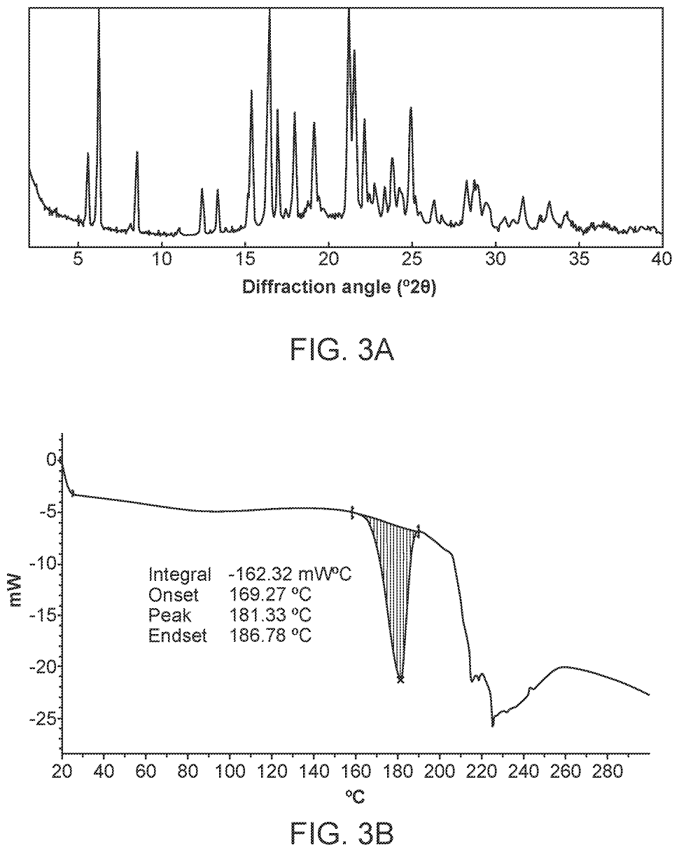

In some embodiments, the diffraction pattern for Compound I Form IIIA is substantially as shown in FIG. 3A.

In some embodiments, a differential scanning calorimetry (DSC) curve for Compound I Form IIIA shows an endotherm onset at about 169.3° C.

In some embodiments, the DSC curve for Compound I Form IIIA is substantially as shown in FIG. 3B.

In some embodiments Compound I Form IIIA is prepared by a process comprising
contacting a solution of Compound I in a solvent with a solution of methanesulfonic acid in a solvent at room temperature to obtain a slurry;
filtering the solids from the slurry to obtain a filter-cake; and
drying the filter-cake under to obtain Compound I Form IIIA.

In some embodiments, the solvent is acetonitrile. In some embodiments, the solvent is isopropanol. In some embodiments, Compound I Form IIA is prepared by the sequence of steps described in the Examples section. In some embodiments, where the solvent was anisole, a similar process provided Compound I Form IIIB. In some embodiments, where the solvent was butanol, 1,4-dioxane, ethyl acetate, methyl ethylketone or toluene, a similar process provided Compound I Form IIIC. In some embodiments, where the solvent was trifluorotoluene or isopropanol, a similar process provided Compound I Form IIID. In some embodiments, Compound I Form IIIB, IIIC, IIID are prepared by the sequence of steps described in the Examples section.

### Compound I Form IVA-IVB—Esylate

In one embodiment, provided herein is crystalline 4-[5-[(3S)-3-aminopyrrolidine-1-carbonyl]-2-[2-fluoro-4-(2-hydroxy-2-methylpropyl)phenyl]phenyl]-2-fluoro-benzonitrile esylate (Compound I Form IVA), characterized by an X-ray powder diffraction pattern comprising the following peaks: 6.17±0.2°, 16.86°±0.2° and 20.92±0.2°, expressed as 2θ angles determined using Cu K-α (λ=1.5406 Å) radiation.

In some embodiments, the diffraction pattern for Compound I Form IVA further comprises one or more peaks selected from 8.42°±0.2°, 18.99°±0.2°, and 21.57°±0.2°, expressed as 2θ angles determined using Cu K-α (λ=1.5406 Å) radiation.

In some embodiments, the diffraction pattern for Compound I Form IVA further comprises two or more peaks selected from 8.420±0.2°, 18.99°±0.2°, 21.57°±0.2°, and 24.26°±0.2°, expressed as 2θ angles determined using Cu K-α (λ=1.5406 Å) radiation.

In some embodiments, the diffraction pattern for Compound I Form IVA comprises peaks selected from 6.17°±0.2°, 8.42°±0.2°, 16.86°±0.2°, 18.99°±0.2°, 20.92°±0.2°, 21.57°±0.2°, and 24.26°±0.2°, expressed as 2θ angles determined using Cu K-α (=1.5406 Å) radiation.

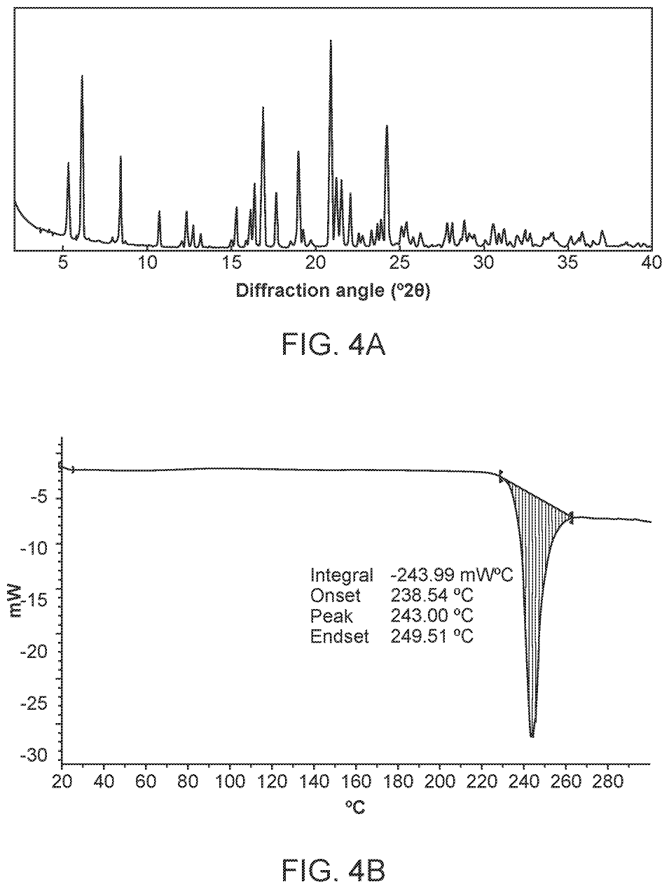

In some embodiments, the diffraction pattern for Compound I Form IVA is substantially as shown in FIG. 4A.

In some embodiments, a differential scanning calorimetry (DSC) curve for Compound I Form IVA shows an endotherm onset at about 238.5° C.

In some embodiments, the DSC curve for Compound I Form IVA is substantially as shown in FIG. 4B.

In some embodiments, Compound I Form IVA is prepared by a process comprising
contacting a solution of Compound I in a solvent with a solution of ethanesulfonic acid in a solvent at room temperature to obtain a slurry;
filtering the solids from the slurry to obtain a filter-cake; and
drying the filter-cake to obtain Compound I Form IVA.

In some embodiments, the solvent is selected from acetonitrile, anisole, butanol, methyl tert-butyl ether (MTBE), ethanol, ethyl acetate, heptane, isopropyl acetate, methyl acetate, methyl ethyl ketone (MEK), and toluene. In some embodiments, Compound I Form IVA is prepared by the sequence of steps described in the Examples section. In some embodiments, where the solvent was THF or isopropanol, a similar process provided Compound I Form IVB. In some embodiments, Compound I Form IVB is prepared by the sequence of steps described in the Examples section.

### Compound I Form V—Maleate

In one embodiment, provided herein is crystalline 4-[5-[(3S)-3-aminopyrrolidine-1-carbonyl]-2-[2-fluoro-4-(2-hydroxy-2-methylpropyl)phenyl]phenyl]-2-fluoro-benzonitrile maleate (Compound I Form V), characterized by an X-ray powder diffraction pattern of Compound I Form IIIA comprising the following peaks: 6.18°±0.2°, 17.41°±0.2° and 19.34°±0.2°, expressed as 2θ angles determined using Cu K-α (λ=1.5406 Å) radiation.

In some embodiments, the diffraction pattern further for Compound I Form V further comprises one or more peaks selected from 18.10°±0.2°, 22.20°±0.2°, and 24.14°±0.2°, expressed as 2θ angles determined using Cu K-α (λ=1.5406 Å) radiation.

In some embodiments, the diffraction pattern further for Compound I Form V further comprises two or more peaks selected from 12.78°±0.2°, 18.10°±0.2°, 22.20°±0.2°, 24.14°±0.2°, and 25.87°±0.2°, expressed as 2θ angles determined using Cu K-α (λ=1.5406 Å) radiation.

In some embodiments, the diffraction pattern further for Compound I Form V comprises peaks selected from 6.18°±0.2°, 12.78°±0.2°, 17.41°±0.2°, 18.10°±40.2°, 19.34°±0.2°, 22.20°±0.2°, 24.14°±40.2°, and 25.87°±0.2°, expressed as 2θ angles determined using Cu K-α (λ=1.5406 Å) radiation.

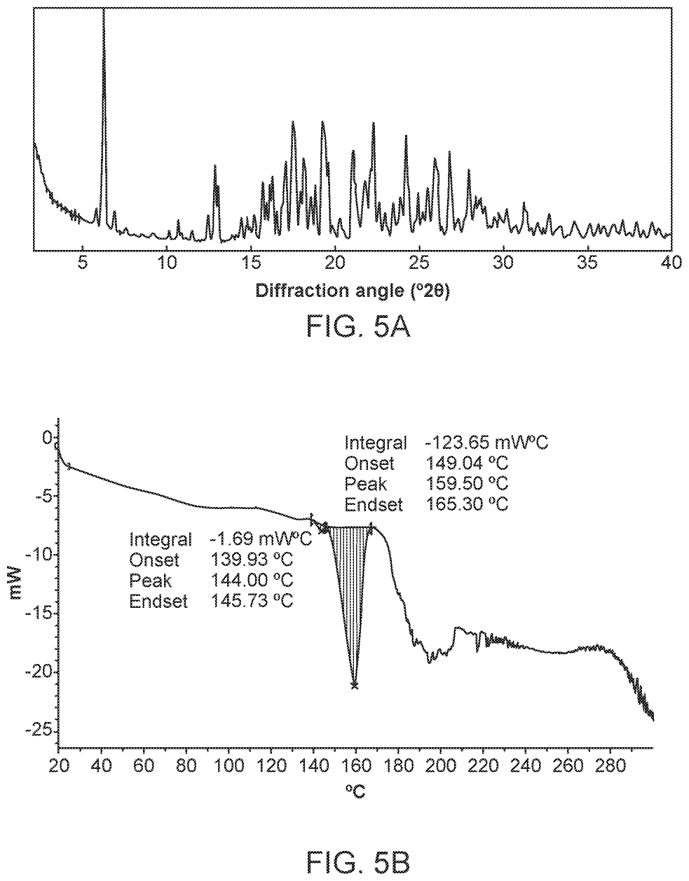

In some embodiments, the diffraction pattern for Compound I Form V is substantially as shown in FIG. 5A.

In some embodiments, a differential scanning calorimetry (DSC) curve for Compound I Form V shows an endotherm onset at about 139.9° C.

In some embodiments the DSC curve for Compound I Form V is substantially as shown in FIG. 5B.

In some embodiments, Compound I Form V is prepared by a process comprising
contacting a solution of Compound I in a solvent with a solution of maleic acid in a solvent at room temperature to obtain a slurry;
filtering the solids from the slurry to obtain a filter-cake; and
drying the filter-cake to obtain Compound I Form V.

In some embodiments, the solvent is selected from acetonitrile, anisole, butanol, isopropanol, methyl tert-butyl ether (MTBE), ethanol, ethyl acetate, heptane, isopropyl acetate, methyl acetate, methyl ethyl ketone (MEK), and toluene. In some embodiments, the solvent is isopropanol. In some embodiments, Compound I Form V is prepared by the sequence of steps described in the Examples section.

### 3. Pharmaceutical Compositions, Kits, and Modes of Administration

The forms of Compound I as described herein may be administered in a pharmaceutical composition. Thus, provided herein are pharmaceutical compositions comprising one or more of the forms of Compound I described herein and one or more pharmaceutically acceptable vehicles such as carriers, adjuvants and excipients. Suitable pharmaceutically acceptable vehicles may include, for example, inert solid diluents and fillers, diluents, including sterile aqueous solution and various organic solvents, permeation enhancers, solubilizers and adjuvants. Such compositions are prepared in a manner well known in the pharmaceutical art. See, e.g., Remington's Pharmaceutical Sciences, Mace Publishing Co., Philadelphia, Pa. 17th Ed. (1985); and Modem Pharmaceutics, Marcel Dekker, Inc. 3rd Ed. (G. S. Banker & C. T. Rhodes, Eds.). The pharmaceutical compositions may be administered alone or in combination with other therapeutic agents.

Some embodiments are directed to pharmaceutical compositions comprising a therapeutically effective amount of a solid form of Compound I described herein. In some embodiments, a pharmaceutical composition comprises a solid form selected from Compound I Form I, Compound I Form II, Compound I Form IIIA, Compound I Form IVA, and/or Compound I Form V; and one or more pharmaceutically acceptable carriers.

Some embodiments are directed to pharmaceutical compositions comprising a crystalline form or amorphous form of Compound I as described herein and one or more pharmaceutically acceptable carriers. In one embodiment, a pharmaceutical composition comprises Compound I, wherein at least 95% of Compound I is in a crystalline form as described herein. In one embodiment, a pharmaceutical composition comprises Compound I, wherein at least 95% of Compound I is in an amorphous form as described herein. In one embodiment, a pharmaceutical composition comprises Compound I, wherein at least 95% of Compound I is in Form I. In one embodiment, a pharmaceutical composition comprises Compound I, wherein at least 95% of Compound I is in Form II. In one embodiment, a pharmaceutical composition comprises Compound I, wherein at least 95% of Compound I is in Form IIIA. In one embodiment, a pharmaceutical composition comprises Compound I, wherein at least 95% of Compound I is in Form IVA. In one embodiment, a pharmaceutical composition comprises Compound I, wherein at least 95% of Compound I is in Form V.

In one embodiment, a pharmaceutical composition comprises Compound I, wherein at least 97% of Compound I is in a crystalline form as described herein. In one embodiment, a pharmaceutical composition comprises Compound I, wherein at least 97% of Compound I is in Form I. In one embodiment, a pharmaceutical composition comprises Compound I, wherein at least 97% of Compound I is in Form II. In one embodiment, a pharmaceutical composition comprises Compound I, wherein at least 97% of Compound I is in Form IIIA. In one embodiment, a pharmaceutical composition comprises Compound I, wherein at least 97% of Compound I is in Form IVA. In one embodiment, a pharmaceutical composition comprises Compound I, wherein at least 97% of Compound I is in Form V.

In one embodiment, a pharmaceutical composition comprises Compound I, wherein at least 99% of Compound I is in a crystalline form as described herein. In one embodiment, a pharmaceutical composition comprises Compound I, wherein at least 99% of Compound I is in Form I. In one embodiment, a pharmaceutical composition comprises Compound I, wherein at least 99% of Compound I is in Form II. In one embodiment, a pharmaceutical composition comprises Compound I, wherein at least 99% of Compound I is in Form IIIA. In one embodiment, a pharmaceutical composition comprises Compound I, wherein at least 99% of Compound I is in Form IVA. In one embodiment, a pharmaceutical composition comprises Compound I, wherein at least 99% of Compound I is in Form V.

In one embodiment, a pharmaceutical composition comprises Compound I, wherein at least 99.5% of Compound I is in a crystalline form as described herein. In one embodiment, a pharmaceutical composition comprises Compound I, wherein at least 99.5% of Compound I is in Form I. In one embodiment, a pharmaceutical composition comprises Compound I, wherein at least 99.5% of Compound I is in Form II. In one embodiment, a pharmaceutical composition comprises Compound I, wherein at least 99.5% of Compound I is in Form IIIA. In one embodiment, a pharmaceutical composition comprises Compound I, wherein at least 99.5% of Compound I is in Form IVA. In one embodiment, a pharmaceutical composition comprises Compound I, wherein at least 99.5% of Compound I is in Form V.

In one embodiment, a pharmaceutical composition comprises Compound I, wherein at least 99.9% of Compound I is in a crystalline form as described herein. In one embodiment, a pharmaceutical composition comprises Compound I, wherein at least 99.9% of Compound I is in Form I. In one embodiment, a pharmaceutical composition comprises Compound I, wherein at least 99.9% of Compound I is in Form II. In one embodiment, a pharmaceutical composition comprises Compound I, wherein at least 99.9% of Compound I is in Form IIIA. In one embodiment, a pharmaceutical composition comprises Compound I, wherein at least 99.9% of Compound I is in Form IVA. In one embodiment, a pharmaceutical composition comprises Compound I, wherein at least 99.9% of Compound I is in Form V.

Any crystalline form of Compound I described herein may be processed, after being pulverized or without being pulverized, into various forms of pharmaceutical composition, for example tablets, capsules, granules, fine granules, powdered drug, dry syrup and like oral preparations, suppositories, inhalation agents, nasal drops, ointments, patches, aerosols, and the like.

In some embodiments, compositions comprise pharmaceutically acceptable carriers or excipients, such as fillers, binders, disintegrants, glidants, lubricants, complexing agents, solubilizers, and surfactants, which may be chosen to facilitate administration of the compound by a particular route. Examples of carriers include calcium carbonate, calcium phosphate, various sugars such as lactose, glucose, or sucrose, types of starch, cellulose derivatives, gelatin, lipids, liposomes, nanoparticles, and the like. Carriers also include physiologically compatible liquids as solvents or for suspensions, including, for example, sterile solutions of water for injection (WFI), saline solution, dextrose solution, Hank's solution, Ringer's solution, vegetable oils, mineral oils, animal oils, polyethylene glycols, liquid paraffin, and the like. Excipients may also include, for example, colloidal silicon dioxide, silica gel, talc, magnesium silicate, calcium silicate, sodium aluminosilicate, magnesium trisilicate, powdered cellulose, macrocrystalline cellulose, carboxymethyl cellulose, cross-linked sodium carboxymethylcellulose, sodium benzoate, calcium carbonate, magnesium carbonate, stearic acid, aluminum stearate, calcium stearate, magnesium stearate, zinc stearate, sodium stearyl fumarate, syloid, stearowet C, magnesium oxide, starch, sodium starch glycolate, glyceryl monostearate, glyceryl dibehenate, glyceryl palmitostearate, hydrogenated vegetable oil, hydrogenated cotton seed oil, castor seed oil mineral oil, polyethylene glycol (e.g. PEG 4000-8000), polyoxyethylene glycol, poloxamers, povidone, crospovidone, croscarmellose sodium, alginic acid, casein, methacrylic acid divinylbenzene copolymer, sodium docusate, cyclodextrins (e.g. 2-hydroxypropyl-.delta.-cyclodextrin), polysorbates (e.g. polysorbate 80), cetrimide, TPGS (d-alpha-tocopherol polyethylene glycol 1000 succinate), magnesium lauryl sulfate, sodium lauryl sulfate, polyethylene glycol ethers, di-fatty acid ester of polyethylene glycols, or a polyoxyalkylene sorbitan fatty acid ester (e.g., polyoxyethylene sorbitan ester Tween®), polyoxyethylene sorbitan fatty acid esters, sorbitan fatty acid ester, e.g. a sorbitan fatty acid ester from a fatty acid such as oleic, stearic or palmitic acid, mannitol, xylitol, sorbitol, maltose, lactose, lactose monohydrate or lactose spray dried, sucrose, fructose, calcium phosphate, dibasic calcium phosphate, tribasic calcium phosphate, calcium sulfate, dextrates, dextran, dextrin, dextrose, cellulose acetate, maltodextrin, simethicone, polydextrosem, chitosan, gelatin, HPMC (hydroxypropyl methyl celluloses), HPC (hydroxypropyl cellulose), hydroxyethyl cellulose, and the like.

Pharmaceutical formulations may be presented in unit dose forms containing a predetermined amount of active ingredient per unit dose. Such a unit may contain, for example, 0.5 mg to 1 g, preferably 1 mg to 700 mg, more preferably 5 mg to 100 mg of a compound of the present disclosure (as a free-acid, solvate (including hydrate) or salt, in any form), depending on the condition being treated, the route of administration, and the age, weight and condition of the patient. Preferred unit dosage formulations are those containing a daily dose, weekly dose, monthly dose, a sub-dose or an appropriate fraction thereof, of an active ingredient. Furthermore, such pharmaceutical formulations may be prepared by any of the methods well known in the pharmacy art.

Compound I, and any of its forms as described herein, are usually administered in the form of pharmaceutical compositions. Thus, provided herein are also pharmaceutical compositions that contain one or more of Compound I, and any of its forms as described herein and one or more pharmaceutically acceptable vehicles selected from carriers, adjuvants and excipients. Suitable pharmaceutically acceptable vehicles may include, for example, inert solid diluents and fillers, diluents, including sterile aqueous solution and various organic solvents, permeation enhancers, solubilizers and adjuvants. Such compositions are prepared in a manner well known in the pharmaceutical art. See, e.g., Remington's Pharmaceutical Sciences, Mace Publishing Co., Philadelphia, Pa. 17th Ed. (1985); and Modern Pharmaceutics, Marcel Dekker, Inc. 3rd Ed. (G. S. Banker & C. T. Rhodes, Eds.).

The pharmaceutical compositions may be administered in either single or multiple doses. The pharmaceutical composition may be administered by various methods including, for example, rectal, buccal, intranasal and transdermal routes. In certain embodiments, the pharmaceutical composition may be administered by intra-arterial injection, intravenously, intraperitoneally, parenterally, intramuscularly, subcutaneously, orally, topically, or as an inhalant.

One mode for administration is parenteral, for example, by injection. The forms in which the pharmaceutical compositions described herein may be incorporated for administration by injection include, for example, aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

When an injection is prepared, a pH adjuster, a buffer, a stabilizer, an isotonizing agent, a topical anesthetic, and the like may be added, as necessary, to the crystalline form of Compound I; and the resulting mixture may be formulated into subcutaneous, intramuscular, and intravenous injections according to an ordinary method.

Examples of usable pH adjusters and buffers include sodium citrate, sodium acetate, sodium phosphate, and the like. Examples of usable stabilizers include sodium pyrosulfite, EDTA, thioglycolic acid, and thiolactic acid. Examples of usable topical anesthetics include procaine hydrochloride, lidocaine hydrochloride, and the like. Examples of usable isotonizing agents include sodium chloride, glucose, D-mannitol, glycerin, and the like.

Oral administration may be another route for administration of the compounds described herein. Administration may be via, for example, capsule or enteric coated tablets. In making the pharmaceutical compositions that include at least one compound described herein or a pharmaceutically acceptable salt, isotopically enriched analog, stereoisomer, mixture of stereoisomers or prodrug thereof, the active ingredient is usually diluted by an excipient and/or enclosed within such a carrier that can be in the form of a capsule, sachet, paper or other container. When the excipient serves as a diluent, it can be in the form of a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments, soft and hard gelatin capsules, sterile injectable solutions, and sterile packaged powders.

Oral solid preparations may be prepared as follows. After an excipient is added optionally with a binder, disintegrant, lubricant, colorant, taste-masking or flavoring agent, etc., to a crystalline form of Compound I, the resulting mixture is formulated into tablets, coated tablets, granules, powders, capsules, or the like by methods known in the art.

Examples of excipients include lactose, sucrose, D-mannitol, glucose, starch, calcium carbonate, kaolin, microcrystalline cellulose, and silicic acid anhydride. Examples of binders include water, ethanol, 1-propanol, 2-propanol, simple syrup, liquid glucose, liquid α-starch, liquid gelatin, D-mannitol, carboxymethyl cellulose, hydroxypropyl cellulose, hydroxypropyl starch, methyl cellulose, ethyl cellulose, shellac, calcium phosphate, polyvinylpyrrolidone, and the like. Examples of disintegrators include dry starch, sodium alginate, powdered agar, sodium hydrogen carbonate, calcium carbonate, sodium lauryl sulfate, stearic acid monoglyceride, lactose, and the like. Examples of lubricants include purified talc, stearic acid salt sodium, magnesium stearate, borax, polyethylene glycol, and the like. Examples of colorants include titanium oxide, iron oxide, and the like. Examples of taste-masking or flavoring agents include sucrose, bitter orange peel, citric acid, L-tartaric acid, and the like. The formulations can additionally include wetting agents; emulsifying and suspending agents; preserving agents such as methyl and propylhydroxy-benzoates.

When a liquid preparation for oral administration is prepared, a taste-masking agent, a buffer, a stabilizer, a flavoring agent, and the like may be added to one of forms of Compound I described herein and the resulting mixture may be formulated into an oral liquid preparation, syrup, elixir, etc., according to an ordinary method.

In this case, the same taste-masking or flavoring agent as those mentioned above may be used. Examples of the buffer include sodium citrate and the like, and examples of the stabilizer include tragacanth, gum arabic, gelatin, and the like. As necessary, these preparations for oral administration may be coated according to methods known in the art with an enteric coating or other coating for the purpose of, for example, persistence of effects. Examples of such coating agents include hydroxypropyl methylcellulose, ethyl cellulose, hydroxymethyl cellulose, hydroxypropyl cellulose, polyoxyethylene glycol, and Tween 80®.

The compositions that include at least one of the forms of Compound I as described herein, can be formulated so as to provide quick, sustained or delayed release of the active ingredient after administration to the subject by employing procedures known in the art. Controlled release drug delivery systems for oral administration include osmotic pump systems and dissolutional systems containing polymer-coated reservoirs or drug-polymer matrix formulations. Another formulation for use in the methods disclosed herein employ transdermal delivery devices (“patches”). Such transdermal patches may be used to provide continuous or discontinuous infusion of the compounds described herein in controlled amounts. The construction and use of transdermal patches for the delivery of pharmaceutical agents is well known in the art. Such patches may be constructed for continuous, pulsatile, or on demand delivery of pharmaceutical agents.

For preparing solid compositions such as tablets, the principal active ingredient may be mixed with a pharmaceutical excipient to form a solid preformulation composition containing a homogeneous mixture of Compound I, and any of its forms as described herein. When referring to these preformulation compositions as homogeneous, the active ingredient may be dispersed evenly throughout the composition so that the composition may be readily subdivided into equally effective unit dosage forms such as tablets, pills and capsules.

The tablets or pills of Compound I, and any of its forms as described herein, may be coated or otherwise compounded to provide a dosage form affording the advantage of prolonged action, or to protect from the acid conditions of the stomach. For example, the tablet or pill can include an inner dosage and an outer dosage component, the latter being in the form of an envelope over the former. The two components can be separated by an enteric layer that serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

In another aspect, the present disclosure provides kits or containers that include a Compound I, and any of its forms as described herein, or any of the pharmaceutical compositions thereof described herein. In some embodiments, the compound or composition is packaged, e.g., in a vial, bottle, flask, which may be further packaged, e.g., within a box, envelope, or bag; the compound or composition is approved by the U.S. Food and Drug Administration or similar regulatory agency for administration to a mammal, e.g., a human; the compound or composition is approved for administration to a mammal, e.g., a human, for a bromodomain protein mediated disease or condition; the kit or container disclosed herein may include written instructions for use and/or other indication that the compound or composition is suitable or approved for administration to a mammal, e.g., a human, for a bromodomain-mediated disease or condition; and the compound or composition may be packaged in unit dose or single dose form, e.g., single dose pills, capsules, or the like.

The amounts of various compounds to be administered can be determined by standard procedures taking into account factors such as the compound activity (in vitro, e.g. the compound IC₅₀ vs. target, or in vivo activity in animal efficacy models), pharmacokinetic results in animal models (e.g. biological half-life or bioavailability), the age, size, and weight of the subject, and the disorder associated with the subject. The importance of these and other factors are well known to those of ordinary skill in the art. Generally, a dose will be in the range of about 0.01 to 50 mg/kg, also about 0.1 to 20 mg/kg of the subject being treated. Multiple doses may be used.

The amount of any form of Compound I to be incorporated in each of such dosage unit forms depends on the condition of the patient to whom the compound is administered, the dosage form, etc. In general, in the case of an oral agent, an injection, and a suppository, the amount of the compound of the present disclosure is preferably 0.05 to 1000 mg, 0.01 to 500 mg, and 1 to 1000 mg, respectively, per dosage unit form.

The daily dose of the medicine in such a dosage form depends on the condition, body weight, age, gender, etc., of the patient, and cannot be generalized. For example, the daily dose of a salt of Compound I described herein for an adult (body weight: 50-70 kg kg) may be 0.05 to 5000 mg, or 0.1 to 1000 mg; and may be administered in one dose, or in two to four divided doses, per day, or any other suitable dosing schedule.

### 4. Dosing

The specific dose level of Compound I, and any of its forms as described herein, for any particular subject will depend upon a variety of factors including the activity of the specific compound employed, the age, body weight, general health, sex, diet, time of administration, route of administration, and rate of excretion, drug combination and the severity of the particular disease in the subject undergoing therapy. For example, a dosage may be expressed as a number of milligrams of a compound described herein per kilogram of the subject's body weight (mg/kg). Dosages of between about 0.1 and 150 mg/kg may be appropriate. In some embodiments, about 0.1 and 100 mg/kg may be appropriate. In other embodiments a dosage of between 0.5 and 60 mg/kg may be appropriate. In some embodiments, a dosage of from about 0.0001 to about 100 mg per kg of body weight per day, from about 0.001 to about 50 mg of compound per kg of body weight, or from about 0.01 to about 10 mg of compound per kg of body weight may be appropriate. Normalizing according to the subject's body weight is particularly useful when adjusting dosages between subjects of widely disparate size, such as occurs when using the drug in both children and adult humans or when converting an effective dosage in a non-human subject such as dog to a dosage suitable for a human subject.

### 5. Disease Indications and Modulation of LSD1

Provided herein is a method for treating a lysine-specific histone demethylase 1A (LSD-1) related disease or condition in a mammal, the method comprising administering to the mammal a therapeutically effective amount of a crystalline form of Compound I described herein, or a composition described herein.

In some embodiments, the LSD-1 related disease or condition is cancer.

In some embodiments, the cancer is a malignant tumor.

In some embodiments, the cancer is head and neck cancer, esophagus cancer, gastric cancer, colon cancer, rectum cancer, liver cancer, gallbladder cancer, cholangiocarcinoma, biliary tract cancer, pancreatic cancer, lung cancer, breast cancer, ovarian cancer, cervical cancer, endometrial cancer, renal cancer, bladder cancer, prostate cancer, testicular tumor, osteosarcoma, soft-tissue sarcoma, leukemia, myelodysplastic syndrome, chronic myeloproliferative disease, malignant lymphoma, multiple myeloma, skin cancer, brain tumor, or mesothelioma.

In some embodiments, the cancer is non-small cell lung cancer, small cell lung cancer, leukemia, or myelodysplastic syndromes.

In certain embodiments, the present disclosure provides the use of Compound I, and any of its forms as described herein, or any of the pharmaceutical compositions thereof described herein in the manufacture of a medicament for the treatment of a disease or condition as described herein. In other embodiments, the present disclosure provides Compound I, and any of its forms as described herein, or any of the pharmaceutical compositions thereof described herein for use in treating a disease or condition as described herein.

### Examples

### Instrumental Techniques

### X-Ray Powder Diffraction

X-Ray powder diffraction (XRPD) analysis was carried out using a Bruker D2 Phaser powder diffractometer equipped with a LynxEye detector. The specimens underwent minimum preparation but, if necessary they were lightly milled in a pestle and mortar before acquisition. The specimens were located at the center of a silicon sample holder within a 5 mm pocket (ca. 5 to 10 mg). The samples were continuously spun during data collection and scanned using a step size of 0.02° two theta (29) between the range of 4° to 40° two theta. Data was acquired using either 3 minute or 20 minute acquisition methods. Data was processed using Bruker Diffrac.Suite.

### Differential Scanning Calorimetry and Thermogravimetric Analysis

A Perkin Elmer Pyris Diamond TG/DTA 6300 was used to measure the weight loss as a function of temperature from 30 to 600° C. The scan rate was 10° C. per minute and the purge gas was nitrogen. A Mettler Toledo DSC 821 instrument was used for the DSC analysis operating with STARe software. The analysis was conducted in 40 μL open aluminum pans, under nitrogen and sample sizes ranged from 1 to 10 mg. Typical analysis method was 20° C. to 250° C. at 10° C./minute.

### Polarised Light Microscopy

The instrument used for digital capture was an Olympus BX41 microscope with digital camera attachment The magnification was ×100 and ×400. Samples were observed under plane polarised and cross polarised light.

### Example 1. Synthesis of Compound I

4-[5-[(3S)-3-aminopyrrolidine-1-carbonyl]-2-[2-fluoro-4-(2-hydroxy-2-methyl-propyl)phenyl]phenyl]-2-fluoro-benzonitrile (Compound I) was prepared according to the procedure described in Example 37 of WO 2017/090756.

### Example 2. Salt Screen

Compound I free base was dispensed into vials. Stock solutions of various acid counter-ions were prepared in methanol, and contained 1.0 eq. of counter-ion, corrected for water contents and assay value, and a stoichiometric amount (1.0 eq or 0.5 eq as applicable) was charged to the vials. The sealed vials were shaken for about 1 to 2 h in order to assist in the dissolution of the substrate and formation of salts. The vials were then oven dried at 40° C. under reduced pressure for about 20 h to ensure the removal of the solvents. The appropriate quench solvents selected for the screen were then added to each vial. The vials were sealed and shaken for about 18 to 20 h. Each vial was inspected for solid formation. Those vials that contained a suitable quantity of solid were centrifuged and the supernatant was decanted off. The solid pellet was dried under reduced pressure for about 20 h and analyzed by XRPD.

Solvents tested included and were not limited to acetone, CAN, TBME, MEK, IPA, IPAc, 1-butanol, methanol, ethanol, DCM, toluene, anisole, ethyl acetate, THF, and/or combinations thereof. Acid counterions tested included and were not limited to sulfate, phosphate, tartrate, fumarate, malate, succinate, glutarate, oxalate, methanesulfonate, ethanesulfonate, lactate, succinate and/or other similar counterions.

### Example 3. Compound I Form I (hemi-fumarate)

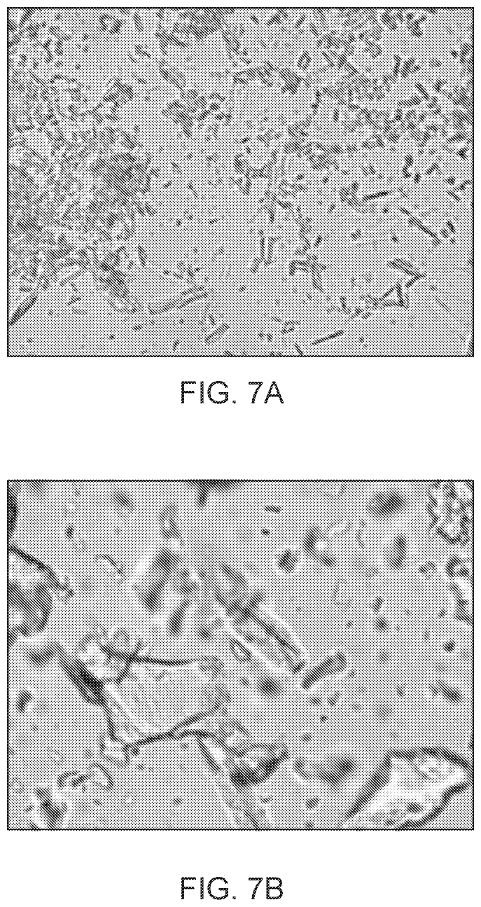

The hemi-fumarate salt (2:1 ratio of Compound I:fumaric acid) Compound I Form I was prepared as follows. To a solution of Compound I freebase (475 mg, 1.0 mmol, 1.0 equiv) in IPA (10 mL) at room temperature was added fumaric acid (58 mg, 0.5 mmol, 0.5 equiv) in IPA (5 mL), and the resulting slurry was warmed up to 50° C. The resulting cloudy slurry was slowly cooled to RT over a period of 2 hours. Then the slurry was stirred for additional 4 hours at room temperature and filtered. The filter-cake was washed with IPA (2 mL) and dried under vacuum at 40° C. for 18 hours to give 300 mg (53%) of Compound I Form I. Form I exhibited packed laths morphology and was highly crystalline. FIG. 1A, FIG. 1B and FIG. 7A show the XRPD, the DSC, and the PLM image respectively for Compound I Form I.

### Example 4. Compound I Form II (Mono-Oxalate)

The mono-oxalate salt (1:1 ratio of Compound I:oxalic acid) was prepared as follows. To a solution of Compound I free base (475 mg, 1.0 mmol, 1.0 equiv) in 2-propanol (10 mL) at room temperature was added oxalic acid (100 mg, 1.1 mmol, 1.1 equiv) in 2-propanol (4 mL), and the resulting cloudy slurry was warmed up to 50° C. The slurry was slowly cooled to RT over a period of 2 hours. The slurry was stirred for additional 4 hours at room temperature and filtered. The filter-cake was washed with cold 2-propanol (5 mL) and dried under vacuum at 40° C. for 42 hours to give 280 mg (49.5%) of Compound I mono-oxalate salt.

Recrystallization of Compound I mono-oxalate salt to provide Form II: Compound I mono-oxalate salt (100 mg, 0.17 mmol) was suspended in ethanol:water (3:2 ratio, 10 mL). The resulting suspension was heated to 80° C. to a clear solution. The clear solution was cooled to room temperature and kept for 2 days. During this period product crystallized out as white solid. The resulting solid separated by filtration and the filter-cake was washed with ethanol:water (3:2 ratio, 2 mL). The compound was dried under vacuum at 40° C. for 18 hours to give 80 mg (80%, recovery) of Compound I Form II. Form II exhibited stacked layers plate-like morphology and was highly crystalline. FIG. 2A, and FIG. 7B show the XRPD, and the PLM image respectively for Compound I Form II.

### Example 5. Compound I Form IIIA-IIID (Mesylate)

To a solution of Compound I free base (250 mg, 0.52 mmol, 1.0 equiv) in IPA (5 mL) (dispensed into 25-mL round-bottom flask) at room temperature was added a solution of the methanesulfonic acid (55.6 mg, 0.57 mmol, 1.1 equiv) in 2-propanol (5 mL). During addition, product/salt started to crystallize out, and the resulting slurry was stirred for another 4 h at room temperature. The product was separated by filtration and the filter-cake was washed with cold 2-propanol (5 mL). The resulting wet-cake was oven-dried under reduced pressure over 72 h at 40° C. Yield: 246 mg, 82% of Compound I Form IIIA. Form IIIA exhibited clustered laths, plates and spherules morphology. FIG. 3A and FIG. 3B show the XRPD, and the DSC respectively for Compound I Form IIIA.

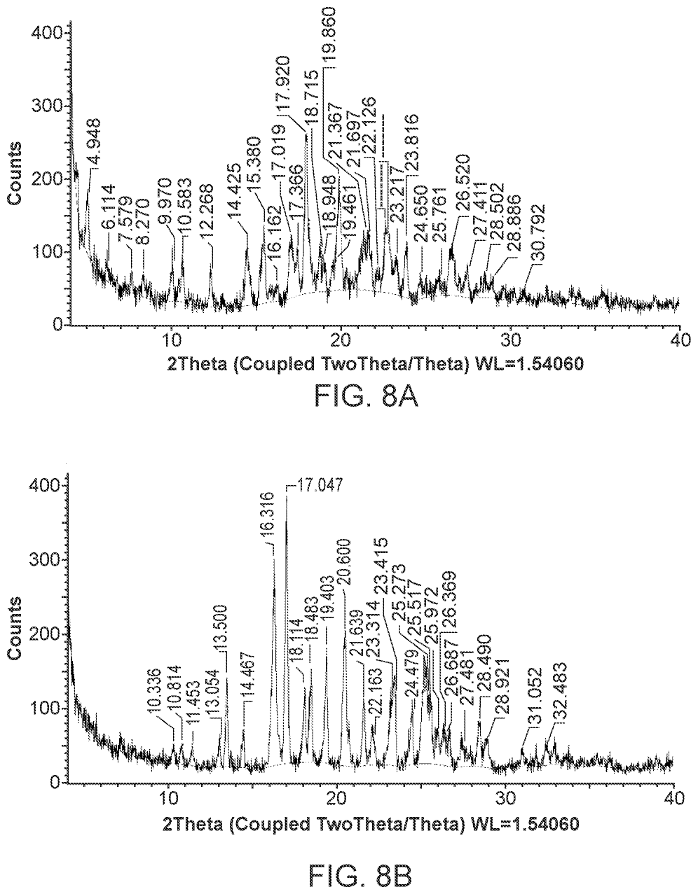

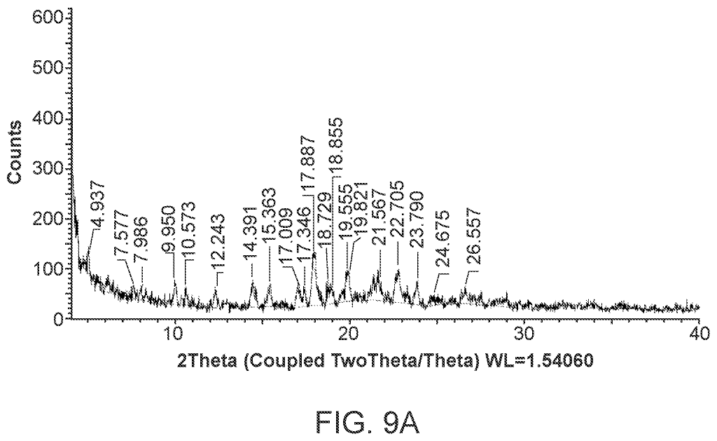

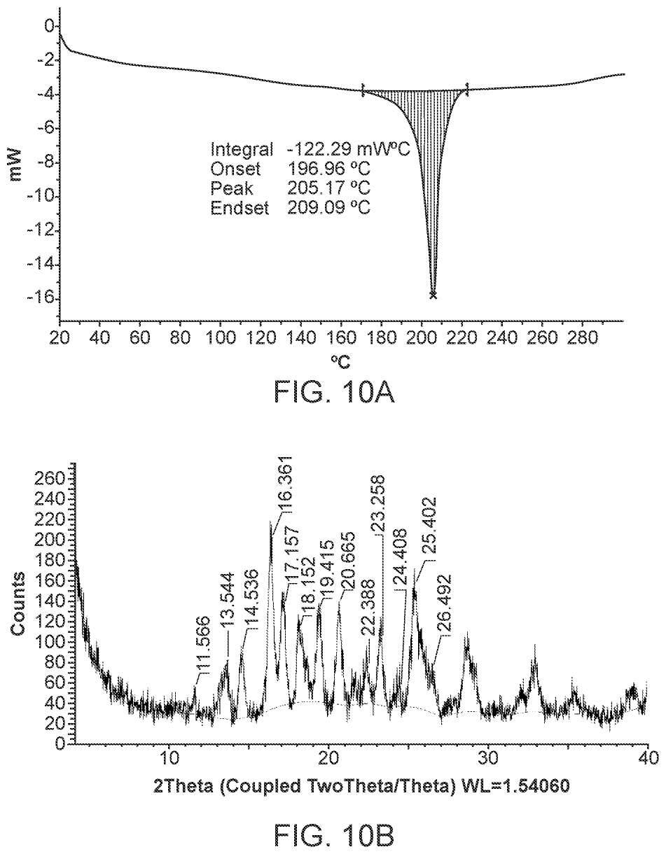

To explore polymorphism of the mesylate, amorphous Compound I mesylate was prepared by mixing compound I (5 g, 10.4 mmol, 1.0 equiv) together with methanesulfonic acid (1.11 g, 11.4 mmol, 1.1 equiv) in methanol (50 mL, 10 vol). The mixture was concentrated to dryness under reduced pressure and further dried under reduced pressure at 40° C. Amorphous Compound I mesylate (about 50 mg), was then mixed with the appropriate solvent (1 mL, 20 vol) and stirred for 7-10 days at 40° C. The mixture was cooled, isolated by filtration and washed with the filtration mother liquors and dried under reduced pressure at 40° C. Compound I Form IIIA was obtained when the solvent was acetonitrile. Compound I form IIIB was obtained when the solvent was anisole. Compound I form IIIC was obtained when the solvent was butanol, 1,4-dioxane, ethyl acetate, methyl ethylketone and toluene. Under these conditions, Compound I form IIID was obtained when the solvent was trifluorotoluene or isopropanol. FIG. 8A and FIG. 8B and FIG. 9A show the XRPDs for Compound I Form IIIB, Compound I Form IIIC and Compound I Form IIID respectively.

### Example 6. Compound I Form IVA-IVB (Esylate)

To a solution of Compound I free base (250 mg, 0.52 mmol, 1.0 equiv) in IPA (5 mL) (dispensed into 25-mL round-bottom flask) at room temperature was added a solution of the ethanesulfonic acid (63.7 mg, 0.57 mmol, 1.1 equiv) in 2-propanol (5 mL). During addition, product/salt started to crystallize out, and the resulting slurry was stirred for another 4 h at room temperature. The product was separated by filtration and the filter-cake was washed with cold 2-propanol (5 mL). The resulting wet-cake oven-dried under reduced pressure over 72 h at 40° C. Yield: 240 mg, 78% of Compound I Form IVA. Form IVA exhibited acicular foliated and folded into tubules morphology. FIG. 4A and FIG. 4B show the XRPD, and the DSC respectively for Compound I Form IVA.

To explore polymorphism of the esylate salt, crystallization from a variety of solvents was carried out. About 50 mg portions of the esylate salt were charged to separate scintillation vials. To each vial was added the appropriate solvent as shown in the table below and the suspensions were heated until full dissolution occurred. If dissolution was not achieved, a binary solvent (either water or methanol) was added in small aliquots at 80° C. until dissolution was achieved. Stirring was suspended and the solutions were removed from the heat, allowed to cool slowly and left to stand undisturbed. The vials that showed crystals were centrifuged and the supernatant was decanted and the solid pellet was oven dried at 40° C. under reduced pressure over about 20 hours.

It was found that using THF as a solvent provided Compound I Form IVB. FIG. 10A and FIG. 10B show the DSC and XRPD respectively for Compound I Form IVB. Compound I Form IVB shows an onset of melt event at about 196° C. versus onset of melt at about 238° C. for Compound I Form IVA.

| Solvent | Yield | XRPD |
|---|---|---|
| Acetone | | |
| Anisole | | |
| Butanol | 81% | Form IVA |
| sec-Butanol | 82% | Form IVA |
| Butyl acetate | | |
| Cumene | | |
| Ethanol | | |
| Ethyl acetate | | |
| Isobutyl acetate | | |
| Isopropyl acetate | 5% | Form IVA |
| Methyl acetate | | |
| 3-methyl-1-butanol | 84% | Form IVA |
| Methyl ethyl ketone | | |
| 2-Methyl-1-propanol | 80% | Form IVA |
| Pentanol | 86% | Form IVA |
| Propanol | 57% | Form IVA |
| Propyl acetate | | |
| THF | 90% | Form IVB (1.4% w.w THF) |
| Acetonitrile | 83% | Form A |
| Chlorobenzene | | |
| DCM | 75% | Disordered (DCM 0.3% w/w) |
| Nitromethane | 86% | Form IVA |
| Toluene | | |

Blank entry indicates no crystals observed

### Example 7. Compound I Form V (Maleate)

To a solution of Compound I free base (250 mg, 0.52 mmol, 1.0 equiv) in IPA (5 mL) (dispensed into 25-mL round-bottom flask) at room temperature was added a solution of the maleic acid (67.2 mg, 0.57 mmol, 1.1 equiv) in 2-propanol (5 mL). During addition, product/salt started to crystallize out, and the resulting slurry was stirred for another 4 h at room temperature. The product was separated by filtration and the filter-cake was washed with cold 2-propanol (2 mL). The resulting wet-cake oven-dried under reduced pressure over 72 h at 40° C. Yield: 217 mg (70%) of Compound I Form V. Form V exhibited packed laths morphology. FIG. 5A and FIG. 5B show the XRPD, and the DSC respectively for Compound I Form V.

### Example 8. Compound I Form VI (Mono-Fumarate)

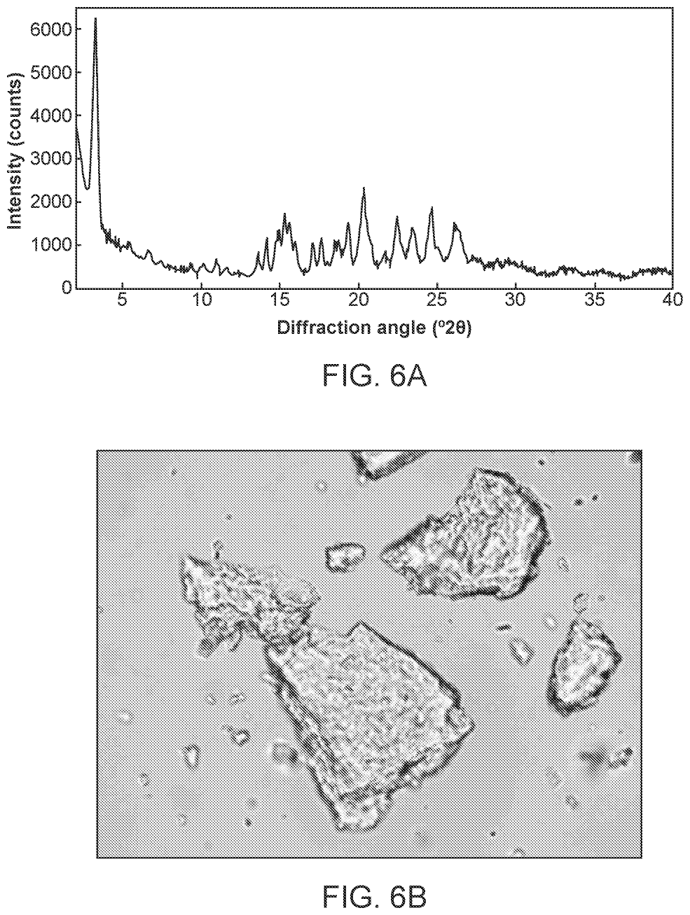

To a solution of Compound I freebase (250 mg, 0.52 mmol, 1.0 equiv) in IPA (5 mL) at room temperature was added a solution of fumaric acid (67.1 mg, 0.57 mmol, 1.1 equiv) in IPA (5 mL), and the resulting slurry was warmed up to 50° C. The resulting cloudy slurry was slowly cooled to RT over a period of 2 hours. Then the slurry was stirred for additional 4 hours at room temperature and filtered. The filter-cake was washed with IPA (5 mL) and dried under vacuum at 40° C. for 72 hours. Yield: 233 mg, 75% of Compound I Form VI. Form VI exhibited packed laths highly aggregated layered crystals morphology. FIG. 6A shows the XRPD for Compound I Form VI.

### Example 9. Compound I Form VII (Hemi-Oxalate)

To a solution of Compound I freebase (475 mg) in 2-propanol or ethanol (10 mL) at room temperature was added oxalic acid (0.45 eq) in 2-propanol or ethanol, and the resulting slurry was warmed up to 50° C. The slurry was slowly cooled to RT over a period of 2 hours and stirred for an additional 4 hours at room temperature, and filtered. The filter cake was washed with cold 2-propanol (5 vol) and dried under vacuum at 45° C. for 18 hours to provide Compound I Form VII. Form VII exhibited semi-crystalline morphology. FIG. 6B shows the PLM image for Compound I Form VII.

### Example 10. Stability Testing

The stability of different salt forms was tested. The salts were placed in 20-mL scintillation vials and covered with Kim wipe, and the resulting vials were exposed to 40° C. and 75% RH (relative humidity) for five days. The samples were analyzed for water absorption. The results are shown below.

| Salt | Initial KF | 5 days at 40° C./75% RH |
|---|---|---|
| mono-fumarate Form VI | 0.61% | 0.63% |
| hemi-fumarate Form I | 0.69% | 1.57% |
| mono-oxalate salt (from IPA) | 2.66% | 4.99% |
| mono-oxalate (from EtOH) Form II | 1.06% | 4.46% |
| mesylate Form IIIA | 2.55% | NT |
| esylate Form IVA | 0.76% | NT |
| maleate Form V | 1.08% | NT |

NT means not tested

The mono-fumarate salt Form VI did not take-up moisture under the tested conditions. The hemi-fumarate (Form I) and the mono-oxalate (Form II) were observed to absorb between 1 and 4.5% of water under the tested conditions.

Both the mono fumarate salt (Form VI) and the hemi fumarate salt (Form I) of Compound I crystallized out of solutions. The mono fumarate salt (Form VI) is a sticky solid and took a long time to filter. The mono-fumarate salt (Form VI) produced plate-like crystals; however, the degree of crystallinity was low by XRPD.

The hemi fumarate salt (Form I) filtered well, and produced tightly packed laths or needles with good crystallinity by XRPD.

Both the mono-oxalate salt and the hemi-oxalate salt (Compound I Form VII) were crystallized out of solutions. The hemi oxalate salt (Form VII) was sticky and took a long time to filter.

The mono-oxalate salt filtered well. However, the mono-oxalate salt obtained from IPA had a low degree of crystallinity by XRPD and was a semi-crystalline material.

The mono-oxalate salt when subsequently recrystallized from ethanol (Compound I Form II) filtered well, and was highly crystalline (XRPD). The particles of the mono-oxalate salt (Form II) appeared to be composed of stacked layers and plate-like morphology.

Overall, the hemi-fumarate Form I was crystalline (XRPD), had low residual water, had a particle size distribution (PSD) of d90=143 μm, and was slightly hygroscopic. Form I exhibited tightly packed laths or needle-like morphology. The mono-oxalate salt recrystallized from ethanol (Form II) was crystalline (XRPD), had PSD of d90=50 μm, had good aqueous solubility, and was slightly hygroscopic. Form II exhibited stacked layers and plate-like morphology.

All patents and other references cited in the specification are indicative of the level of skill of those skilled in the art to which the disclosure pertains, and are incorporated by reference in their entireties, including any tables and figures, to the same extent as if each reference had been incorporated by reference in its entirety individually.

One skilled in the art would readily appreciate that the present disclosure is well adapted to obtain the ends and advantages mentioned, as well as those inherent therein. The methods, variances, and compositions described herein as presently representative of preferred embodiments are exemplary and are not intended as limitations on the scope of the disclosure. Changes therein and other uses will occur to those skilled in the art, which are encompassed within the spirit of the disclosure, are defined by the scope of the claims.

What is claimed is:

1. Crystalline 4-[5-[(3S)-3-aminopyrrolidine-1-carbonyl]-2-[2-fluoro-4-(2-hydroxy-2-methylpropyl)phenyl]phenyl]-2-fluoro-benzonitrile hemi fumarate (Compound I Form I), characterized by an X-ray powder diffraction pattern comprising the following peaks: 9.37°±0.2°, 14.63°±0.2° and 21.27°±0.2°, expressed as 2θ angles determined using Cu K-α (λ=1.5406 Å) radiation.

2. The crystalline form of claim 1, wherein the diffraction pattern further comprises one or more peaks selected from 4.73°±0.2°, 18.86°±0.2°, and 20.65°±0.2°, expressed as 2θ angles determined using Cu K-α (λ=1.5406 Å) radiation.

3. The crystalline form of claim 1, wherein the diffraction pattern further comprises two or more peaks selected from 4.73°±0.2°, 8.18°±0.2°, 14.79°±0.2°, 18.86°±0.2°, 20.41°±0.2° and 20.65°±0.2°, expressed as 2θ angles determined using Cu K-α (λ=1.5406 Å) radiation.

4. The crystalline form of any one of claims 1-3, wherein the diffraction pattern is substantially as shown in FIG. 1A.

5. The crystalline form of any one of claims 1-4, wherein the crystalline form is prepared by a process comprising
contacting a solution of Compound I in a solvent with a solution of fumaric acid in a solvent at room temperature to obtain a slurry;
warming the slurry to a temperature of about 50° C.;
cooling the slurry to room temperature;
filtering the solids from the slurry to obtain a filter-cake; and
drying the filter-cake to obtain Compound I Form I.

6. The process of claim 5, wherein the solvent is selected from acetonitrile, anisole, butanol, isopropanol, methyl tert-butyl ether (MTBE), ethanol, ethyl acetate, heptane, isopropyl acetate, methyl acetate, methyl ethyl ketone (MEK), and toluene.

7. The process of claim 5, wherein the solvent is isopropanol.

8. Crystalline 4-[5-[(3S)-3-aminopyrrolidine-1-carbonyl]-2-[2-fluoro-4-(2-hydroxy-2-methylpropyl)phenyl]phenyl]-2-fluoro-benzonitrile mono-oxalate (Compound I Form II), characterized by an X-ray powder diffraction pattern comprising the following peaks: 13.86°±0.2°, 19.05°±0.2° and 22.94°±0.2°, expressed as 2θ angles determined using Cu K-α (λ=1.5406 Å) radiation.

9. The crystalline form of claim 8, wherein the diffraction pattern further comprises one or more peaks selected from 18.50°±0.2°, 22.31°±0.2°, and 28.48°±0.2°, expressed as 2θ angles determined using Cu K-α (λ=1.5406 Å) radiation.

10. The crystalline form of claim 8, wherein the diffraction pattern further comprises two or more peaks selected from 15.83°±0.2°, 16.32°±0.2°, 18.50°±0.2°, 18.79°±0.2°, 22.31°±0.2°, and 28.48°±0.2°, expressed as 2θ angles determined using Cu K-α (λ=1.5406 Å) radiation.

11. The crystalline form of any one of claims 8-10, wherein the diffraction pattern is substantially as shown in FIG. 2.

12. The crystalline form of any one of claims 8-11, wherein the crystalline form is prepared by a process comprising
contacting a solution of Compound I in a solvent with a solution of oxalic acid in a solvent at room temperature to obtain a slurry;
warming the slurry to a temperature of about 50° C.;
cooling the slurry to room temperature;
filtering the solids from the slurry to obtain a filter-cake;
drying the filter-cake to obtain Compound I mono-oxalate salt; and
recrystallizing the Compound I mono-oxalate salt from ethanol to obtain Compound I Form II.

13. The process of claim 12, wherein the solvent is selected from acetonitrile, anisole, butanol, isopropanol, methyl tert-butyl ether (MTBE), ethanol, ethyl acetate, heptane, isopropyl acetate, methyl acetate, methyl ethyl ketone (MEK), and toluene.

14. The process of claim 12, wherein the solvent is isopropanol.

15. Crystalline 4-[5-[(3S)-3-aminopyrrolidine-1-carbonyl]-2-[2-fluoro-4-(2-hydroxy-2-methylpropyl)phenyl]phenyl]-2-fluoro-benzonitrile mesylate (Compound I Form IIIA), characterized by an X-ray powder diffraction pattern comprising the following peaks: 6.24°±0.2°, 16.47°±0.2° and 21.20°±0.2°, expressed as 2θ angles determined using Cu K-α (λ=1.5406 Å) radiation.

16. The crystalline form of claim 15, wherein the diffraction pattern further comprises one or more peaks selected from 15.39°±0.2°, 16.97°±0.2°, and 21.51°±0.2°, expressed as 2θ angles determined using Cu K-α (λ=1.5406 Å) radiation.

17. The crystalline form of claim 16, wherein the diffraction pattern further comprises two or more peaks selected from 8.52°±0.2°, 15.39°±0.2°, 16.97°±0.2°, 17.95°±0.2°, and 21.51°±0.2°, expressed as 2θ angles determined using Cu K-α (λ=1.5406 Å) radiation.

18. The crystalline form of any one of claims 15-17, wherein the diffraction pattern is substantially as shown in FIG. 3A.

19. The crystalline form of any one of claims 15-18, characterized by a differential scanning calorimetry (DSC) curve that shows an endotherm onset at about 169.3° C.

20. The crystalline form of claim 19, wherein the DSC curve is substantially as shown in FIG. 3B.

21. The crystalline form of any one of claims 15-20, wherein the crystalline form is prepared by a process comprising
contacting a solution of Compound I in a solvent with a solution of methanesulfonic acid in a solvent at room temperature to obtain a slurry;
filtering the solids from the slurry to obtain a filter-cake; and
drying the filter-cake under to obtain Compound I Form IIIA.

22. The process of claim 21, wherein the solvent is acetonitrile or isopropanol.

23. The process of claim 21, wherein the solvent is isopropanol.

24. Crystalline 4-[5-[(3S)-3-aminopyrrolidine-1-carbonyl]-2-[2-fluoro-4-(2-hydroxy-2-methylpropyl)phenyl]phenyl]-2-fluoro-benzonitrile esylate (Compound I Form IVA), characterized by an X-ray powder diffraction pattern comprising the following peaks: 6.17°±0.2°, 16.86°±0.2° and 20.92°±0.2°, expressed as 2θ angles determined using Cu K-α (λ=1.5406 Å) radiation.

25. The crystalline form of claim 24, wherein the diffraction pattern further comprises one or more peaks selected from 8.42°±0.2°, 18.99°±0.2°, and 21.57°±0.2°, expressed as 2θ angles determined using Cu K-α (λ=1.5406 Å) radiation.

26. The crystalline form of claim 24, wherein the diffraction pattern further comprises two or more peaks selected from 8.42°±0.2°, 18.99°±0.2°, 21.57°±0.2°, and 24.26°±0.2°, expressed as 2θ angles determined using Cu K-α (λ=1.5406 Å) radiation.

27. The crystalline form of any one of claims 25-26, wherein the diffraction pattern is substantially as shown in FIG. 4A.

28. The crystalline form of any one of claims 24-27, characterized by a differential scanning calorimetry (DSC) curve that shows an endotherm onset at about 238.5° C.

29. The crystalline form of claim 28, wherein the DSC curve is substantially as shown in FIG. 4B.

30. The crystalline form of any one of claims 24-29, wherein the crystalline form is prepared by a process comprising
contacting a solution of Compound I in a solvent with a solution of ethanesulfonic acid in a solvent at room temperature to obtain a slurry;
filtering the solids from the slurry to obtain a filter-cake; and
drying the filter-cake to obtain Compound I Form IVA.

31. The process of claim 30, wherein the solvent is selected from acetonitrile, anisole, butanol, methyl tert-butyl ether (MTBE), ethanol, ethyl acetate, heptane, isopropyl acetate, methyl acetate, methyl ethyl ketone (MEK), and toluene.

32. The process of claim 30, wherein the solvent is isopropanol.

33. Crystalline 4-[5-[(3S)-3-aminopyrrolidine-1-carbonyl]-2-[2-fluoro-4-(2-hydroxy-2-methylpropyl)phenyl]phenyl]-2-fluoro-benzonitrile maleate (Compound I Form V), characterized by an X-ray powder diffraction pattern of Compound I Form IIIA comprising the following peaks: 6.18°±0.2°, 17.41°±0.2° and 19.34°±0.2°, expressed as 2θ angles determined using Cu K-α (λ=1.5406 Å) radiation.

34. The crystalline form of claim 33, wherein the diffraction pattern further comprises one or more peaks selected from 18.10°±0.2°, 22.20°±0.2°, and 24.14°±0.2°, expressed as 2θ angles determined using Cu K-α (λ=1.5406 Å) radiation.

35. The crystalline form of claim 33, wherein the diffraction pattern further comprises two or more peaks selected from 12.78°±0.2°, 18.10°±0.2°, 22.20°±0.2°, 24.14°±0.2°, and 25.870±0.20, expressed as 2θ angles determined using Cu K-α (λ=1.5406 Å) radiation.

36. The crystalline form of any one of claims 33-35, wherein the diffraction pattern is substantially as shown in FIG. 5A.

37. The crystalline form of any one of claims 33-36, characterized by a differential scanning calorimetry (DSC) curve that shows an endotherm onset at about 139.9° C.

38. The crystalline form of claim 37, wherein the DSC curve is substantially as shown in FIG. 5B.

39. The crystalline form of any one of claims 33-38, wherein the crystalline form is prepared by a process comprising
contacting a solution of Compound I in a solvent with a solution of maleic acid in a solvent at room temperature to obtain a slurry;
filtering the solids from the slurry to obtain a filter-cake; and
drying the filter-cake to obtain Compound I Form V.

40. The process of claim 39, wherein the solvent is selected from acetonitrile, anisole, butanol, isopropanol, methyl tert-butyl ether (MTBE), ethanol, ethyl acetate, heptane, isopropyl acetate, methyl acetate, methyl ethyl ketone (MEK), and toluene.

41. The process of claim 39, wherein the solvent is isopropanol.

42. The crystalline form of any one of claims 1-41, wherein the crystalline form is in a substantially pure form.

43. A pharmaceutical composition comprising a crystalline form of Compound I of any one of claims 1 to 42, and one or more pharmaceutically acceptable carriers.

44. The pharmaceutical composition of claim 43, wherein at least 99% of the Compound I is in a crystalline from.

45. The pharmaceutical composition of claim 43, wherein at least 95% of the Compound I is in a crystalline from.

46. A method for treating a lysine-specific histone demethylase 1A (LSD-1) related disease or condition in a mammal, the method comprising administering to the mammal a therapeutically effective amount of the crystalline form of any one of claims 1-42, or the composition of claim 43.

47. The method of claim 46, wherein the LSD-1 related disease or condition is cancer.

48. The method of claim 47, wherein the cancer is a malignant tumor.

49. The method of claim 47, wherein the cancer is head and neck cancer, esophagus cancer, gastric cancer, colon cancer, rectum cancer, liver cancer, gallbladder cancer, cholangiocarcinoma, biliary tract cancer, pancreatic cancer, lung cancer, breast cancer, ovarian cancer, cervical cancer, endometrial cancer, renal cancer, bladder cancer, prostate cancer, testicular tumor, osteosarcoma, soft-tissue sarcoma, leukemia, myelodysplastic syndrome, chronic myeloproliferative disease, malignant lymphoma, multiple myeloma, skin cancer, brain tumor, or mesothelioma.

50. The method of claim 47, wherein the cancer is non-small cell lung cancer, small cell lung cancer, leukemia, or myelodysplastic syndromes.
